SEQUENCE LISTING (9 sequences)

SEQ ID NO: 1 - DNA, 22 bp, Homo sapiens
cggaaactgttagatgcaagag

SEQ ID NO: 2 - DNA, 20 bp, Homo spaiens
ctggtgccttcatcttggac

SEQ ID NO: 3 - DNA, 23 bp, Homo sapiens
gactctcttacagtgaggagcac

SEQ ID NO: 4 - DNA, 23 bp, Homo sapiens
tcttcacaggattcatctaaagc

SEQ ID NO: 5 - DNA, 19 bp, Homo sapiens
cagacggaaccatggaagc

SEQ ID NO: 6 - DNA, 20 bp, Homo sapiens
taacactctgactggccctg

SEQ ID NO: 7 - DNA, 482 bp, Homo sapiens
tttttttttttttttttcccagacaagaaaaaattatattttgcaaggttttgaatgagaacacgcctccaaatcaaacatatgtgttgtcttaatgattaaaatagtctcttgttttacaaaggcagatttgatctttctatcaaataaagcatgttttgatgattaaaatgtttgtaaacacagcagtagtcagaaagaacactttcatttgttgaatccatttttcctctatctcttctttcctttcttccttgagttgcccataagggccaggcaggtggtgcaaaatagcggaaactgttagatgcaagagtagggatgctggggtggtgatgaactggagagtgcgggctccatagttccgaaggctgggaagtccaagatgaaggcaccagcaggtttgttgcctggtgagggcccagtgtttgctttcatgaacactgcatcctcaggagggaggaacactgtgacctcacatg

SEQ ID NO: 8 - DNA, 1271 bp, Homo sapiens
ctgtattgtggttcctggaacactttagaggcttgtgattctactgcttcttattcacactataatacatgtctcaccaatagatgattcaagaacatcatttaaatacacaatttttcattctctatttttgctaaatttcttcatactcaactttcagattctttaatctccagctcagcttcaacaattcaacgctgttctttctgaaaaagtacacatcgtgccttctctacttcgctcttggaacataatttctcatggcagcttttactaaactgagtattgagccagcatttactccaggacccaacatagaactccagaaagactctgactgctgttcttgccaagaaaaatgggttgggtaccggtgcaactgttacttcatttccagtgaacagaaaacttggaacgaaagtcggcatctctgtgcttctcagaaatccagcctgcttcagcttcaaaacacagatgaactggattttatgagctccagtcaacaattttactggattggactctcttacagtgaggagcacaccgcctggttgtgggagaatggctctgcactctcccagtatctatttccatcatttgaaacttttaatacaaagaactgcatagcgtataatccaaatggaaatgctttagatgaatcctgtgaagataaaaatcgttatatctgtaagcaacagctcatttaaatgtttcttggggcagagaaggtggagagtaaagacccaacattactaacaatgatacagttgcatgttatattattactaattgtctacttctggagtctataaaatgtttttaaacagtgtcatatacaattgtcatgtatgtgaaacaatgtgttttaaaattgatgaaattcgttcacctacatttgagaattataaaattaacataaagaattttgtattttcatttaatgtatataatgttaaattcaatgtagttttattacacatttatgtaattttatttacattcttgctaattctcagcagaaatttaaataagatttaattcacatcaaataaaatttagaaaataaaatttaactcacactgcccaggctggagcatagtggcaagatcatagctcattgcaagctcaagtgatcctcctgactcagcctcccaagtagctaggactgcaggcaccatgtcactatgcccgactaatttttaatttttaattttttgtcaagacaaggtcttgctatgttgcccaggctggtcttgaactcctggcctcaa

SEQ ID NO: 9 - DNA, 459 bp, Homo sapiens
cagttaggacccagacggaaccatggaagccccagcgcagcttctcttcctcctgctactctggctcccagacaccactggagaaatagtgatgacgcagtctccagccaccctgtctgtgtctccaggggaaagagccaccctctcctgcagggccagtcacagtgttagcgtcaacttagcctggtaccagcagaaacctggccacactcccaggctcctcatctatggtgcatccacccgggccactggtatcccagccaggttcagtggcagtgggtctgggacagagttcactctcaccatcaacagcctgcagtctgaagattttgcagtttattactgtcaacaatataatgattggcctccgtactcttctggccaggggaccaagctggagatcagacgaactgtggctgcaccatctgtcttcatcttcccgccatctgatgagcagtt

(19) United States
(12) Patent Application Publication
(10) Pub. No.: US 2007/0003946 A1
(43) Pub. Date: Jan. 4, 2007

(54) Use of biomarkers in screening for insulin resistance

(76) Inventors: Yuanhong Ma, Los Altos, CA (US); Xingyu Wang, Beijing (CN); Lisheng Liu, Beijing (CN)

Correspondence Address: LYNN E. MURRY, 424 WEST PATRICIA LNAE, FAYETTEVILLE, AR, 72703, US

(21) Appl. No.: 11/297,512

(22) Filed: Dec. 8, 2005

Related U.S. Application Data
(63) Continuation-in-part of application No. 10/161,803, filed on Jun. 3, 2002, now abandoned.
(60) Provisional application No. 60/295,264, filed on Jun. 1, 2001.

(51) Int. Cl. C12Q 1/68; C07H 21/04

(52) U.S. Cl. 435/6; 536/24.3

(57) ABSTRACT

The invention relates to methods and biomarkers useful in screening subjects for insulin resistance and for monitoring insulin resistance over time or after medical intervention.

### CROSS-REFERENCE TO RELATED APPLICATIONS

This application is a continuation-in-part of U.S. Ser. No. 10/161,803, filed 3 Jun. 2002, incorporated by reference herein in its entirely for all purposes and claims benefit of provisional application Ser. No. 60/295,264, filed 1 Jun. 2001.

### FIELD OF THE INVENTION

The present invention is related to the use of biomarkers in screening for insulin resistance and is used in the fields of biology and medicine.

### BACKGROUND OF THE INVENTION

Glucose homeostasis involves glucose absorption in the gut, glucose utilization by insulin-responsive adipose, brain and muscle tissue, glucose sensing and insulin secretion by the pancreas, and glucose synthesis and storage by the liver (Shepherd and Kahn (1999) New England J Med 341:248-57). Blood glucose levels are regulated by circulating hormones, primarily insulin and glucagon; cellular proteins for insulin signaling and glucose transport, and multiple genetic factors that still need to be identified and studied.

Insulin resistance is a prediabetic condition that describes the decreased ability of peripheral tissue to respond to insulin and import and utilize glucose. If the pancreas is capable of secreting more insulin in response to this condition, normal glucose tolerance can often be maintained by medical intervention including changes in lifestyle (diet, exercise and weight loss) and/or taking over-the-counter or prescription insulin sensitizing drugs. Untreated insulin resistance can progress to metabolic syndrome or syndrome X (Reaven GM (1988) Diabetes 37:1595-1607; Abbasi et al (2002) J Amer College Cardiol 40:937-43). When the pancreas is no longer able to produce insulin, insulin resistance generally progresses to non-insulin dependent diabetes mellitus (type 2 diabetes, NIDDM), and adults with insulin resistance have up to 10 times greater risk of death from cardiovascular diseases and are at greater risk for NIDDM and related problems with eyes, kidneys and nerves (America Diabetes Association (2002; Diabetes Care 25:742-49; 2003; Diabetes Care 26:917-32). According the World Health Organization, NIDDM is now the most costly healthcare issue worldwide as well as a major contributor to mortality and morbidity in developed countries. It should also be noted that those who remain sensitive to insulin as they age have almost zero incidence of age-related diseases such as cancer, coronary heart disease, hypertension, NIDDM, and stroke (Facchini et al (2001) J Clin Endocrin Metabol 86:3574-78).

There is a need in the art for early screening and/or diagnosis of insulin resistance so that subjects can take advantage of a selection of treatment options to prevent progression of insulin resistance to metabolic syndrome and NIDDM.

### SUMMARY OF THE INVENTION

The present invention claims a method of screening for insulin resistance using biomarkers for insulin resistance (BIRs) that are differentially expressed in insulin resistant (IR) subjects. The differential expression of the BIRs in subjects who are IR when compared to an subject who is insulin sensitive or to a standard reference value from population of normal, insulin sensitive individuals is at least two-fold.

The invention provides a combination comprising primer sets selected from biomarkers of insulin resistance (BIRs), wherein three of the primer sets are SEQ ID NOs:1-6 and three of the BIRs are SEQ ID NOs:7-9.

The invention provides a method for screening a subject for insulin resistance comprising obtaining a sample from the subject, extracting mRNA from the sample, performing real time quantitative PCR (RT-qPCR) on the sample using a combination of primer sets selected from BIRs to quantitate gene expression and comparing the amount of gene expression in the sample to the amount of gene expression in a sample from at least one normal subject, wherein differential expression between the samples indicates insulin resistance. In a first aspect of the invention, the samples are blood samples. In a second aspect of the invention, differential expression is at least a two-fold decrease in expression of at least one BIL In a third aspect, expression in a sample from at least one normal age and gender matched subject further comprises a standard reference value for insulin sensitivity.

The invention also provides a method for monitoring IR status in an IR subject comprising obtaining a sample from the subject, extracting mRNA from the sample, performing real time quantitative PCR on the sample using the combination of primer sets selected from BIRs to assess gene expression, and comparing gene expression in the sample to gene expression in a previous sample from the subject, wherein differential expression determines a change in IR status. In one aspect of the invention, differential expression denoting a change in IR status indicates increased glucose tolerance or insulin sensitivity. In another aspect of the invention, differential expression denoting a change in IR status indicates progression of IR In yet another aspect of the invention, differential expression denoting a change in IR status is prognostic for the onset of IR-related conditions and non-insulin dependent diabetes mellitus. In still yet another aspect of the invention, differential expression denoting a change in IR status indicates successful lifestyle intervention. In a further aspect of the invention, differential expression denoting a change in IR status following drug therapy indicates successful therapeutic intervention. In a yet further aspect of the invention, IR-related conditions are selected from metabolic syndrome, obesity, atherosclerosis, peripheral vascular disease, hypertension, myocardial infarction and stroke.

The invention further provides a method for assessing therapeutic efficacy of a drug used to treat insulin resistance in a subject comprising administering a drug to the subject and comparing the amount of expression in a first sample taken from the subject prior to administering the drug to the amount of expression in a second sample taken from the subject following administration of the drug, wherein a significant difference in expression indicates therapeutic efficacy.

The invention additionally provides a combination comprising three BIRs, wherein the BIRs are SEQ ID NOs:7-9 that can be used in methods of screening, or rescreening, for insulin resistance and assessing medical intervention or therapeutic efficacy.

### DESCRIPTION OF THE FIGURES AND TABLES

A portion of the disclosure of this patent document contains material which is subject to copyright protection. The copyright owner has no objection to the facsimile reproduction by anyone of the patent document or the patent disclosure, as it appears in the Patent and Trademark Office patent file or records, but otherwise reserves all copyright rights whatsoever.

The Sequence Listing is a compilation of primer sets, SEQ ID NOs:1-9, and the representative GenBank polynucleotide sequences to which they align, SEQ ID NOs:7-9, respectively.

### DETAILED DESCRIPTION OF THE INVENTION

### DEFINITIONS

It is to be understood that the terminology below is used for the purpose of describing particular embodiments and is not intended to limit the scope of the invention which will be limited only by the appended claims.

“Gene product” refers to mRNA, or a cDNA produced from the mRNA, expressed by a biomarker of the invention.

“Insulin resistant (Related conditions” refers to metabolic syndrome, obesity, and cardiovascular diseases including atherosclerosis, peripheral vascular disease, hypertension, myocardial infarction and stroke.

“Medical intervention” refers to the changes in lifestyle (diet, exercise and weight loss) or use of over-the-counter or prescription drugs to ameliorate symptoms of insulin resistance or slow the progression towards IR-related conditions or non-insulin dependent diabetes mellitus (NIDDM).

“NIDDM” refers to type 2 diabetes as it is characterized by hyperglycemia, occurring from a decrease in insulin sensitivity and/or an increase in insulin resistance, and the failure of increased insulin secretion to compensate for the impaired glucose metabolism as associated with serious neurological, opthalmic, renal and vascular complications that can cause premature death if undiagnosed and untreated.

“OGTT” (oral glucose tolerance test) refers to a test commonly used to determine a subject's ability to produce and utilize insulin to metabolize glucose based on plasma glucose or insulin concentrations in a blood sample at specified time intervals after oral adminisation of 75 grams of (g) glucose.

“Primer” refers a single-stranded oligonucleotide from about 18 to about 30 nucleotides in length which can be used in hybridization or amplification technologies. Equivalent terms include amplimer and oligomer.

“Primer set” refers to forward 5′ and reverse 3′ primers used to produce an “amplicon”, the intervening region of a polynucleotide that the primers border and that is amplified in a polymerase chain reaction (PCR).

“Polynucleotide” refers to an isolated polynucleotide, cDNA, RNA, oligonucleotide or nucleic acid molecule that can have originated naturally, recombinantly or synthetically, be double-stranded or single-stranded, represents coding, intronic, or noncoding 5′ or 3′ sequence, and can be purified from or combined with carbohydrate, lipids, protein or inorganic elements or substances as a useful composition.

“Sample” is used in its broadest sense as containing nucleic acids, proteins, antibodies, and the like and refers to a bodily fluid; the soluble fraction of a cell preparation, or an aliquot of media in which cells were grown; a chromosome, an organelle, or membrane isolated or extract from a cell; genomic DNA, RNA, or cDNA in solution or bound to a substrate; a cell; a tissue or tissue biopsy; a tissue print; buccal cells, a hair follicle, muscle, skin, and the like.

“Specificity” refers to the proportion of non-insulin sensitive or non-insulin resistant subjects who were correctly identified as IR or normal using OGTT.

“Standard” refers to a reference value chosen to be representative of gene expression in a population of individuals of the same gender and of approximately the same age who are known to be glucose tolerant and sensitive to insulin.

“Therapeutic efficacy” refers to that amount of a drug used for medical intervention that will elicit a biological or medical response in a cell, tissue, system, animal or human that is being sought by a medical doctor or other clinician to ameliorate symptoms or to slow or reverse progression toward a particular disease.

### DETAILED DESCRIPTION

The present invention identifies biomarkers for insulin resistance (BIRs) that distinguish subjects who are insulin resistant (prediabetic) from those who are normal (glucose tolerant and sensitive to insulin) based on the differential expression of BIR gene product in a subject sample. The BERs were selected from a larger set of insulin resistance markers (IFRs) differentially expressed in blood samples assayed using microarray technology as described in patent application, U.S. Ser. No. 10/161,803, filed 3 Jun. 2002, and incorporated by reference herein in its entirely for all purposes.

Three primer sets for the BIRs are presented in the sequence listing as SEQ ID NOs: 1-6. These primer sets were selected from sequences with the IRM identifiers and GenBank accession numbers presented in the table below and in U.S. Ser. No. 10/161,803, filed 3 Jun. 2002. The first column shows BIR numbers; the second column, IRM numbers; the third column, GenBank accession numbers; and the fourth column, SEQ ID NOs.

| BIRs | IRMs | GENBANK ACCESSION NOs. | SEQ ID Nos |
|---|---|---|---|
| 1 | 130 | 814057, AW298595 | 1, 2 |
| 2 | 170 | NM_007334, NM_002262 | 3, 4 |
| 3 | 200 | X58082, BC030813 | 5, 6 |

Primers for amplification reactions can be designed using Blast analysis (NCBI, Bethesda Md.), Oligo 6 software (Molecular Biology Insights, Cascade Colo.) or another software program, to be about 18-30 bp in length, to have a GC content of about 50%, to anneal to the RNA or cDNA at temperatures from about 68 C to about 72 C, and when possible, to span an intron/exon splice junction so that the resulting 100-600 bp amplicon can separated from any contaminating genomic DNA in the reaction. Any stretch of nucleotides which would result in hairpin structures and primer-primer dimerizations (also know as primer-dimers) are avoided. It is contemplated that a large number of additional primer sets as well as the cDNAs related to the GenBank sequences as represented by SEQ ID NOs:7-9 could be used in hybridization and amplification methods set forth in the instant invention and the parent application, U.S. Ser. No. 10/161,803.

The original IMAGE clone cDNAs from which the primers were selected are available from Invitrogen (Carlsbad CA) and their nucleotide sequences can be confirmed using routine methods. For experimental use, primers can be synthesized using de novo chemical synthesis (Beaucage et al (2000) Current Protocols in Nucleic Acid Chemistry. John Wiley & Sons, New York N.Y.) or ordered from Invitrogen, MWG Biotech (High Point N.C.) or Sigma-Aldrich (St. Louis Mo.).

About 5000 subjects at three different Beijing hospitals were considered for this study, and 2500 subjects were enrolled. Family history was taken from these 2500 subjects who were between 30 and 65 years of age, had normal thyroid function, were not pregnant, had not been diagnosed with any liver, kidney, heart or infectious disease, and had fasted overnight (˜10-12 hours) prior to testing. As part of an oral glucose tolerance test (OGTT), each subject was given an 75 g glucose orally, and blood samples were taken by venipuncture at 0, 30, 60 and 120 minutes (min) after administration of glucose. In the present invention, all glucose concentrations were reported in mg/dl and insulin concentrations, in μIU/ml. RNA was extracted from the blood of each of the 2500 subjects. Triglyceride, total cholesterol, high density lipoprotein, and low density lipoprotein, alanine aminotransferase, and results of urinalysis were also recorded for each subject (data not shown).

OGIT was used to select 34 age and gender matched subject pairs, one of whom was known to be IR and the other, known to be sensitive to insulin. Gender and age matched pairs were used because the ability to metabolize glucose decreases and the mean concentration of glucose in the blood increases with age and is slightly more pronounced in females (data not shown). The OGTT criteria used for identifying these subjects had a specificity of 95% as shown in Example II. Blood samples taken from the 34 subject pairs were screened for insulin resistance. After red blood cell (RBC) removal, leukocytes were lysed for RNA extrction using the methods and chemicals set forth in Example IV. Both the blood samples and extracted RNA can be subjected to a variety of well-known post-collection preparative and storage techniques (eg, storage in ethanol, freezing, etc) prior to assessing the amount of the BIR expression in the sample.

Because expression levels can vary from tissue to tissue and with changes in other physiological parameters, the samples screened must compare BIR expression based on the same kind of bodily fluid or tissue sampled under the same physiological conditions (ie, fasting and no recent infection that could alter the numbers of white blood cells and their levels of gene expression). For example, when a blood sample is used, the blood will be collected after an overnight fast and compared to subject or standard reference values based on blood samples taken after an overnight fast. A subject can be identified as “at risk” for insulin resistance prior to screening based on family medical history.

Primer sets for BIR1, BIR2, and BIR3 and real time quantitative PCR (RT-qPCR) were used to screen the RNA samples for differential expression. A significant difference in BIR expression between IR and normal samples of at least two-fold is shown in the table in Example IV. At least one primer pair was differentially expressed in 71% of the paired samples demonstrating that BIRs are useful in screening for IR.

The present invention provides a method, BIRs and their primer sets not only to screen subject samples for insulin resistance, but also to monitor subject IR status and prognosis, response to medical intervention, progression toward IR related conditions and NIDDM and therapeutic efficacy.

A subject whi is identified as susceptible to IR based on family medical history or as IR based on results of OGTT or another assay can be rescreened at regular intervals. Readings of BIR expression for the subject can be compared with earlier values from the subject or against standards. The earlier value(s) from screening a subject for IR can be used to establish a baseline value for the subject. Statistical methods can be used to determine and to set standard deviation for baseline values of BIR expression. Medical intervention including changes in lifestyle and/or taking drugs such as insulin sensitizers can increase glucose tolerance and/or insulin sensitivity. A subject with successful intervention will find that expression levels for the BIRs have increased and the development of IR-related conditions and NIDDM has been delayed. Conversely, a subject with a further decrease in expression levels is at increased risk for IR-related conditions and NIDDM. In such cases, the subject's physician can take more aggressive prophylactic measures and also begin screening and/or monitoring the subject for IR-related conditions and conversion to NIDDM.

A standard reference value, a statistical value determined for a population of individuals grouped by age (preferably based on about five year intervals—eg, 36-40 and 41-45 years of age) and gender who have tested normal (ie, glucose tolerant and insulin sensitive) over time and have no apparent risk factors for insulin resistance are useful in screening for subjects with insulin resistance. Standard reference values for BIR expression can be established by taking fasting blood samples from a population of normal, insulin sensitive, human subjects, extracting mRNA from the samples, running RT-qPCR on the samples using the BIR primer sets and determining the amount of expression for each BIR. The expression for each BIR can be analyzed using statistical software to establish a standard reference value for glucose tolerance and/or insulin sensitivity. Standard reference values for age, insulin sensitive, and gender defined populations can replace individual pairs or baseline values to screen subject samples for IR or differential expression over time. Similarly, deviation from the standard reference values toward those associated with either insulin sensitivity or insulin resistance can used to monitor IR status, either progression toward increased glucose tolerance and insulin sensitivity or toward increased glucose intolerance, IR-related conditions and NIDDM.

The method of the invention can also be used to evaluate the efficacy of medical intervention or a particular therapeutic treatment of an individual subject. In these cases, a baseline can be established for the subject prior to therapy and screening repeated on a regular basis after treatment starts. It is important to evaluate whether gene expression levels are moving in the desired direction, toward insulin sensitivity, as a result of the treatment.

### EXAMPLES

It is to be understood that the invention is not limited to the particular methodology, protocols, and reagents described as these can vary. The examples below are provided to illustrate and enable the invention and are not included for the purpose of limiting the invention.

I Measuring Glucose, Insulin and Lipids

Five thousand subjects were screened using OGTT to identify 2500 unrelated, non-diabetic subjects. All subjects in the study had normal thyroid function and were not known to be pregnant or affected by any liver, kidney, heart, or infectious disease. Individuals who had a glucose concentration greater than 126 mg/dl at 0 min or greater than 200 mg/dl at 120 min after glucose administration, triglyceride over 600 mg/dl, total cholesterol greater than 600 mg/dl, systolic blood pressure greater than 170 mmHg, diastolic blood pressure greater than 100 mmHg or BMI greater than 32 kg/m² or less than 18 kg/m², were excluded from the study.

After an overnight fast of 10-12 hours (hr), subjects reported to the hospital and were given 75 g glucose orally (83 g prepared sugar powder containing 75 g D-glucose in 12 ounces of water, LongFu Hospital, Beijing, China). At 0, 30, 60 and 120 min after the administration of glucose, five ml of blood were collected from each subject by venipuncture and placed in sterile test tubes without anticoagulent. The top layer, serum, was collected from each tube after standing for one hr at room temperature. The serum was aliquoted for determination of glucose and insulin concentrations and for extraction of RNA from white blood cells as described below. The study was IRB-approved, and the informed consent of all subjects was obtained prior to participation.

Measurements of Glucose Concentration

All glucose concentrations were analyzed using the standard hexokinase method and Olympus AU2700 Chemistry-Immuno analyzer (Olympus America, Melville N.Y.). To ensure the quality and consistency of the data, the analyzer was calibrated twice a week using standards with known glucose concentrations. OGTT data were analyzed using standard statistical program software (version 10.1; SSPS Inc, Chicago Ill.).

Measurements of Insulin Concentration

All insulin concentrations were analyzed using Coat-A-Count Insulin Radioimmunoassay kits (Diagnostic Products, Los Angeles Calif.). In this solid-phase radioimmunoassay, ¹²⁵I-labeled insulin competes with insulin in the sample for sites on an insulin-specific antibody. All procedures were performed for the fixed time period specified by the manufacturer.

Briefly, 200 μl of serum collected from each subject at each time point was mixed with 1.0 ml of ¹²⁵I-insulin (tracer) in a Coat-A-Count tube pre-coated with fixed amount of insulin-specific antibody. After 18-24 hr incubation at room temperature, the supernatant was decanted; and the tube was allowed to drain for 2-3 min. The radioactivity in the tube was counted for one min using an SN-697 automatic gamma radiation counter (Shanghai He-Suo-Ri-Huan Photoelectric Instrument Ltd., Shanghai, China). Counts were converted to insulin concentration in μIU/ml according to the kit instructions. To ensure data quality and consistency, duplicate sample aliquots were tested against standards and controls provided in the kit by the manufacturer.

II Selection Criteria for Identifying Normal and IR Subjects

The following selection criteria were used to select age (within ±two years) and gender matched subject pairs for the molecular studies. All individuals were between 30 and 65 years of age and had ALT <80 U/L.

| | Min | Normal | IR* |
|---|---|---|---|
| OGTT glucose (mg/dl) | 0 | <126 | <126 |
| ″ | 60 | ≦121 and | >170 and |
| ″ | 120 | ≦100 | >121 and <200 |
| OGTT insulin (μIU/ml) | 60 | ≦50 or | >60 and |
| ″ | 120 | ≦40 | >60 |

*>95% specificity

III Primer Identification and Synthesis

Primers were designed using Oligo 6 software (Molecular Biology Insights, Cascade Colo.) and the GenBank sequences presented in the U.S. Ser. No. 10/161,803 as IRMs 130, 170, and 200, identified by Genbank accession numbers: AW298595, NM_{—}007334, and X58082, respectively. The DNA sequence for each accession number was used perform a BLAT analysis (UCSC Genome Brower on Human, version June 2002) in order to identify longer or full-length cDNAs or genomic sequences and the intron-exon boundaries in the genomic sequence. cDNA sequences flanking an intron were used to design primer sets for specific amplification of the cDNA without amplifying contaminating genomic DNA. The primers were selected to avoid setches of nucleotides that could form dimers in the PCR amplification process. The primers had a melting temperature of about 60 C, were about 20-23 bp in length, and when possible, spanned an intron/exon splice junction so that the resulting 104-159 bp amplicons could be separated from contaminating genomic DNA.

All primers were ordered from Sigma Proligo (formerly Genset; now Sigma-Aldrich). Primer sets corresponding to the SEQ ID NOs:1 and 2 are mapped to IRM-130, GenBank Accession AW298595 (SEQ ID NO:7) as shown highlighted below:

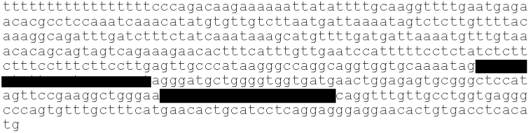

Primer sets corresponding to the SEQ ID NOs:3 and 4 are mapped to IRM-170, GenBank Accession NM_{—}007334 (SEQ ID NO:8) as shown highlighted below:

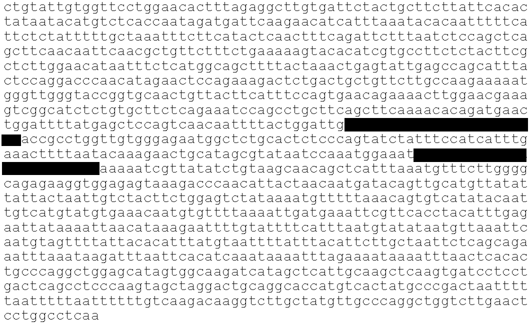

Primer sets corresponding to the SEQ ID NOs:5 and 6 are mapped to IRM-170, GenBank Accession X58082 (SEQ ID NO:9) as shown highlighted below:

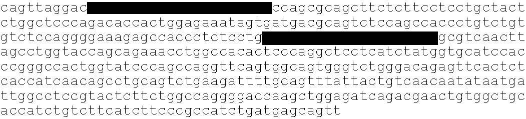

IV RNA Extraction and Real Time Quantitative PCR
RNA extraction

Another 12-15 ml of blood was collected from each subject in a tube containing EDTA and processed for RNA within four hr of collection. The whole blood was centrifuged at 3000 rpm for five min at room temperature in a Sorvall RT 6000D centrifuge (DuPont, Wilmington Del.). Most of the serum was removed, and about 1-1.5 ml of serum from directly above the buffy coat was placed in a 50 ml centrifuge tube and vortexed with 40 ml of red blood cell (RBC) lysis buffer (8.28 g of ammonium chloride, 0.84 g sodium bicarbonate, 500 μl of 0.2 M EDTA (pH 8.0) and distilled water to 1 L). After incubation at room temperature for 15 min, the tube was centrifuged at 2000× g for five min, and the supernatant was discarded. The pellet was resuspended in 15 ml RBC lysis buffer and centrifuged as above to remove any contaminants that could inhibit RT-qPCR. After discarding the supernatant, the pellet was resuspended in about 0.3 ml phosphate buffer solution (PBS) and vortexed at maximum speed for 30 sec. PBS containing the white blood cells was divided into two parts and transferred into two ml Genemate microfuge tubes (1SC Bioexpress, Kaysville Utah). One ml TRizol reagent (Invitrogen, Carlsbad Calif.) was added to each tube to lyse the cells. Then, 0.2 ml chloroform was added to the tube which was shaken vigorously and then placed in a rack at room temperature for 15 min to allow phase separation to occur. The tube was centrifuged in a Beckman J2-21 (Beckman Coulter, Fullerton Calif.) at 14000 rpm for 15 min at room temperature. The upper ⅔ of the aqueous phase was transferred immediately to a fresh 2 ml RNAse-free microfuge tube with a screw cap and silicone O-ring (VWR, West Chester Pa.). An equal volume of isopropanol was added, and the tube was inverted at least 20 times. The tube was placed in the freezer at −20 C for 15 min then centrifuged at 12000 rpm for 15 min. After the tubes were checked for a pellet, they were stored at −20 C.

Measuring Gene Expression for Each BIR using RT-qPCR

In order to measure the expression level of the BIRs, SYBR-green RT-qPCR assays was performed using primer sets specific for BIR1 (forward primer: 5′-CGGAAACTGTTAGATGCAAGA G-3′ (SEQ ID NO:1); reverse primer: 5′-CTGGTGCCTTCATCTTGGAC-3′ (SEQ ID NO:2) generating an amplicon of 104 bp); BIR2 (forward primer: 5′-GACTCTCTTACAGTGAGGAGCAC-3′ (SEQ ID NO:3); reverse primer: 5′-TCTTCACAGGATTCATCTAAAGC-3′ (SEQ ID NO:4) generating an amplicon of 151 bp), and BIR3 (forward primer: 5′-CAGACGGAACCATGGAAGC-3′ (SEQ ID NO:5); reverse primer: 5′-TAACACTCTGACTGGCCCTG-3′ (SEQ ID NO:6) generating an amplicon of 159 bp).

Tubes containing RNA pellets were removed from storage, and the pellets were re-suspended in 100 ul of DEPC-treated Tris buffer (10 mM, pH7.0). cDNA synthesis was performed using approximately 0.5 ug of total RNA from 34 unrelated IR subjects and from 34 age and gender matched insulin sensitive subjects unrelated to IR subjects and each other. First strand synthesis of cDNA was made using SuperScript reverse transcriptase (Invitrogen) with random hexaners. After inactivation of the reverse transcriptase by heat denaturation, the sample was digested by RnaseH to eliminate RNA. The cDNA was then purified away from primers, unreacted dNTPs and enzymes using the Qiaquick DNA purification kit (Qiagen, Valencia Calif.). In order to measure the expression level of the three BIR genes, SYBR-green RT-q PCR assays were performed using primers specific for each of the three BIRs.

Each PCR reaction contained 300 nM of each primer in the primer set, 10 ng of cDNA, and 2× SYBR-green PCR ready mix (Applied Biosystems, Foster City Calif.) in a final volume of 50 μl. The PCR reaction and real time detection was performed using the ABI Prism Sequencing Detection System 7700 (Applied Biosystems). The PCR conditions were set as follows: 95 C for 12 min followed by 40 cycles at 95 C for 30 sec, then at 59 C for 30 sec.

RT-PCR was performed in duplicate on the matched subject pair samples, and data was collected during and at the end of the process. Gene expression levels were analyzed using ABI sequencing detection software, version 1.6 (Applied Biosystems). At the end of each run, the size and quality of each amplicon was verified by examining the disassociation curve and using gel electrophoresis to ensure the absence of non-specific amplification and/or any primer-dimer bands. An aliquot of reaction mixture was loaded into a 3% agarose gel in an electrophoresis tank containing 1× TBE and run at 100 V for 45 min alongside a 1 ug DNA ladder for size standardization (ISC BioExpress).

The expression level of the gene target was translated into Ct (cycle threshold), a user defined threshold at which the fluorescence intensity due to double stranded DNA binding to SYBR-green is 10-fold the background value (determined earlier in the PCR reaction). A higher expression level is translated into an earlier Ct (smaller number), and the converse. Initially, the analyte Ct was normalized versus the control gene, beta actin. The difference between analyte and control Ct, defined as the delta Ct, was compared to control gene values to obtain the ΔΔCt value. In the test range (15-35 Ct), ΔCt represents a two-fold difference, and ΔΔCt can be used to obtain the relative expression of the analyte (ABI user bulletin #2; Applied Biosytems).

Results of Screening Paired Samples

The following exemplary table shows the results of BIR screening. Column 1 shows the matched subject pairs; column 2, the OGTT status (insulin resistant=IR or normal insulin sensitive=IS) for each sample; column 3, the ID assigned to each subject; column 4, the gender of the subject; column 5, the age of each subject; column 6, the differential expression attributable to BIR1; column 7, the differential expression attributable to BIR2; column 8, the differential expression attributable to BIR3; and column 9, the validation of IR.

| | OGTT | | | | | | | Vali- |
|---|---|---|---|---|---|---|---|---|
| Pain | Status | ID | Gender | Age | BIR1 | BIR2 | BIR3 | dation |
| 2.1 | IR | 335 | F | 45 | −6.65 | NS | −3.24 | IR |
| 2.2 | IS | 146 | F | 44 | | | | |
| 4.1 | IR | 211 | F | 51 | NS | NS | NS | IR |
| 4.2 | IS | 251 | F | 51 | | | | |
| 5.1 | IR | 104 | M | 57 | −2.36 | NS | NS | IR |
| 5.2 | IS | 62 | M | 57 | | | | |
| 7.1 | IR | 435 | F | 39 | NS | NS | NS | IR |
| 7.2 | IS | 112 | F | 40 | | | | |
| 8.1 | IR | 796 | M | 39 | NS | NS | −2.00 | IR |
| 8.2 | IS | 64 | M | 39 | | | | |
| 9.1 | IR | 899 | F | 47 | −3.58 | −2.37 | NS | IR |
| 9.2 | IS | 55 | F | 47 | | | | |
| 10.1 | IR | 649 | F | 59 | NS | NS | NS | — |
| 10.2 | IS | 83 | F | 59 | | | | |
| 11.1 | IR | 497 | M | 65 | NS | NS | −3.36 | IR |
| 11.2 | IS | 165 | M | 65 | | | | |
| 12.1 | IR | 528 | F | 48 | NS | NS | −2.54 | IR |
| 12.2 | IS | 181 | F | 47 | | | | |
| 13.1 | IR | 863 | F | 54 | −2.24 | −3.48 | NS | IR |
| 13.2 | IS | 188 | F | 55 | | | | |
| 14.1 | IR | 33 | F | 52 | NS | NS | NS | — |
| 14.2 | IS | 814 | F | 52 | | | | |
| 16.1 | IR | 207 | M | 52 | NS | −3.13 | NS | IR |
| 16.2 | IS | 892 | M | 51 | | | | |
| 17.1 | IR | 391 | F | 45 | −2.38 | NS | NS | IR |
| 17.2 | IS | 938 | F | 45 | | | | |
| 18.1 | IR | 84 | F | 53 | −3.34 | NS | NS | IR |
| 18.2 | IS | 600 | F | 53 | | | | |
| 19.1 | IR | 68 | M | 61 | 2.42 | NS | −3.36 | IR |
| 19.2 | IS | 895 | M | 63 | | | | |
| 20.1 | IR | 103 | F | 60 | NS | −4.00 | NS | IR |
| 20.2 | IS | 802 | F | 58 | | | | |
| 21.1 | IR | 121 | F | 54 | NS | NS | NS | — |
| 21.2 | IS | 881 | F | 54 | | | | |
| 22.1 | IR | 265 | M | 39 | 2.18 | −2.63 | −2.00 | IR |
| 22.2 | IS | 280 | M | 39 | | | | |
| 23.1 | IR | 843 | M | 39 | NS | NS | NS | IR |
| 23.2 | IS | 691 | M | 40 | | | | |
| 24.1 | IR | 731 | M | 45 | NS | NS | NS | IR |
| 24.2 | IS | 871 | M | 44 | | | | |
| 25.1 | IR | 685 | M | 48 | 2.31 | NS | NS | — |
| 25.2 | IS | 850 | M | 49 | | | | |
| 26.1 | IR | 547 | M | 50 | NS | NS | NS | — |
| 26.2 | IS | 399 | M | 50 | | | | |
| 27.1 | IR | 792 | M | 58 | NS | 2.21 | −10.6 | IR |
| 27.2 | IS | 518 | M | 58 | | | | |
| 28.1 | IR | 564 | M | 31 | NS | −3.13 | 3.7 | IR |
| 28.2 | IS | 283 | M | 31 | | | | |
| 29.1 | IR | 659 | M | 35 | NS | −3.73 | −5.65 | IR |
| 29.2 | IS | 355 | M | 36 | | | | |
| 30.1 | IR | 776 | M | 37 | −3.37 | NS | −2.83 | IR |
| 30.2 | IS | 370 | M | 36 | | | | |
| 31.1 | IR | 530 | F | 41 | NS | NS | NS | — |
| 31.2 | IS | 679 | F | 41 | | | | |
| 32.1 | IR | 257 | F | 43 | NS | −4.59 | NS | IR |
| 32.2 | IS | 740 | F | 43 | | | | |
| 33.1 | IR | 225 | F | 46 | NS | −3.61 | NS | IR |
| 33.2 | IS | 559 | F | 46 | | | | |
| 34.1 | IR | 835 | F | 46 | NS | −2.80 | −2.38 | IR |
| 34.2 | IS | 325 | F | 47 | | | | |
| 35.1 | IR | 177 | F | 48 | NS | −2.00 | NS | IR |
| 35.2 | IS | 610 | F | 47 | | | | |
| 36.1 | IR | 827 | F | 48 | NS | −6.73 | NS | IR |
| 36.2 | IS | 369 | F | 48 | | | | |
| 37.1 | IR | 868 | F | 48 | −3.66 | −2.00 | 5.85 | IR |
| 37.2 | IS | 828 | F | 48 | | | | |
| 38.1 | IR | 647 | F | 49 | NS | NS | −2.83 | IR |
| 38.2 | IS | 859 | F | 48 | | | | |

* NS = non-significant

For each BIR marker, the relative level of expression in the IR subject was compared with the relative level of expression in the control IS subject Since the standard deviation of the RT-qPCR system was about 0.4 to 0.5 CT (based on more than 7 repeated quality control assays using a beta-actin reference gene in each sample), 2× standard deviation (or 2×0.5 CT=1 Ct=two-fold difference) was used as the cut-off for significance. For example, ID 2.1 (IR) was underexpressed by 3.24-fold compared to ID 2.2 (IS) for marker BIR3; so −3.24 was listed. However, ID 2.1 (IR) was under-expressed by less than two-fold as compared to ID 2.2 (IS) for BIR2, so NS (non-significant) was listed. If an IR subject sample was overexpressed by more than two-fold (eg, ID 37.1 (IR) for marker BIR3), it was listed as 5.85.

These data confirmed that BIRs were differentially expressed at least two-fold in 71% of known IR subjects. Moreover, 24% of the time, two markers showed differential expression on the same sample. With these data, the method of the invention has been demonstrated to be useful in screening (or rescreening) subjects for insulin resistance, determining prognosis, monitoring medical intervention, and assessing therapeutic efficacy of a drug

V Use of BIRs to Evaluate Medical Intervention

BIR pairs and RT-qPCR can be used to evaluate the medical intervention—suggested changes in lifestyle associated with changes in diet, implementation of exercise and weight loss or administration of a drug to increase glucose tolerance or insulin sensitivity. Baseline BIR expression levels can be established for the subject prior to medical intervention, and the subject can be rescreened regularly to determine if BIR expression has begun to approximate a normal or standard reference value. Rescreening shows whether BIR expression levels are moving toward increased glucose tolerance or insulin sensitivity or the converse as a result of lifestyle intervention or drug therapy.

VI Use of BIRs to Evaluate Therapeutic Efficacy

BIR primer sets and RT-qPCR can be used to evaluate therapeutic efficacy of an over-the-counter or prescription drug that increases glucose tolerance or insulin sensitivity. Baseline BIR expression levels can be established for the subject prior to taking the drug, and the subject can be rescreened regularly to determine if BIR expression has begun to approximate normal expression level or a standard reference value. Rescreening shows whether BIR expression levels are moving toward increased glucose tolerance or insulin sensitivity as a result of taking the drug. In general, an increase in BIR expression levels indicates therapeutic efficacy.

VII Hybridization Technologies

Immobilization of cDNAs on a Substrate

The cDNAs of the invention are applied to a substrate by one of the following methods. A mixture of cDNAs is fractionated by gel electrophoresis and transferred to a nylon membrane by capillary transfer. Alternatively, the cDNAs are individually ligated to a vector and inserted into bacterial host cells to form a library. The cDNAs are then arranged on a substrate by one of the following methods. In the first method, bacterial cells containing individual clones are manually or robotically picked and arranged on a nylon membrane. The membrane is placed on LB agar containing a selective agent (carbenicillin, kanamycin, ampicillin, or chloramphenicol depending on the vector used) and incubated at 37 C for 16 hr. The membrane is removed from the agar and placed colony side up in 10% SDS, denaturing solution (1.5 M NaCl, 0.5 M NaOH ), neutralizing solution (1.5 M NaCl, 1 M Tris, pH 8.0), and twice in 2×SSC for 10 min each. The membrane is then UV irradiated in a STRATALINKER UV-crosslinker (Stratagene, La Jolla Calif.).

In the second method, cDNAs are amplified by thirty cycles of PCR using primers complementary to the vector sequences flanking the insert. PCR amplification increases a starting concentration of 1-2 ng nucleic acid to a final quantity greater than 5 μg. Amplified nucleic acids from about 400 bp to about 5000 bp in length are purified using SEPHACRYL-400 beads (Amersham Biosciences, Piscataway N.J.). Purified nucleic acids are arranged on a nylon membrane manually or using a dot/slot blotting manifold and suction device and are immobilized by denaturation, neutralization, and UV irradiation as described above. Purified nucleic acids are manually or robotically arranged and immobilized on polymer-coated glass slides. Polymer-coated slides are prepared by cleaning glass microscope slides (Coming Life Sciences) by ultrasound in 0.1% SDS and acetone, etching in 4% hydrofluoric acid (VWR Scientific Products), coating with 0.05% aminopropyl silane (Sigma-Aldrich) in 95% ethanol, and curing in a 110 C oven. The slides are washed extensively with distilled water between and after treatments. The nucleic acids are arranged on the slide and then immobilized by exposing the microarray to UV irradiation using a STRATALINKER UV-crosslinker (Stratagene). Microarrays are then washed at room temperature in 0.2% SDS and rinsed three times in distilled water. Non-specific binding sites are blocked by incubation of microarrays in 0.2% casein in PBS for 30 min at 60 C; then the microarrays are washed in 0.2% SDS and rinsed in distilled water as before.

Probe Preparation for Membrane Hybridization

Hybridization probes derived from the cDNAs are employed for screening cDNAs, mRNAs, or genomic DNA in membrane-based hybridizations. Probes are prepared by diluting the cDNAs to a concentration of 40-50 ng in 45 μl TE buffer, denaturing by heating to 100 C for five min, and briefly centrifuging. The denatured cDNA is then added to a REDIPRIME tube (Applied Biosystems), gently mixed until blue color is evenly distributed, and briefly centrifuged. Five μl of [³²P]dCTP is added to the tube, and the contents are incubated at 37 C for 10 min. The labeling reaction is stopped by adding 5 μl of 0.2 M EDTA, and probe is purified from unincorporated nucleotides using a PROBEQUANT G-50 microcolumn (Applied Biosystems). The purified probe is heated to 100 C for five min, snap cooled for two min on ice, and used in membrane-based hybridizations as described below.

Probe Preparation for Polymer Coated Slide Hybridization

Hybridization probes derived from mRNA isolated from samples are employed for screening cDNAs in microarray-based hybridizations. Probe are prepared by diluting mRNA to a concentration of 200 ng in 9 μl TE buffer and adding 5 μl 5× buffer, 1 μl 0.1 M DTT, 3 μl Cy3 or Cy5 labeling mix, 1 μl RNAse inhibitor, 1 μl reverse transcriptase, and 5 μl 1× yeast control mRNAs. Yeast control mRNAs are synthesized by in vitro transcription from noncoding yeast genomic DNA. As quantitative controls, one set of control mRNAs at 0.002 ng, 0.02 ng, 0.2 ng, and 2 ng are diluted into reverse transcription reaction mixture at ratios of 1:100,000, 1:10,000, 1:1000, and 1:100 (w/w) to sample mRNA respectively. To examine mRNA differential expression patterns, a second set of control mRNAs are diluted into reverse transcription reaction mixture at ratios of 1:3, 3:1, 1:10, 10:1, 1:25, and 25:1 (w/w). The reaction mixture is mixed and incubated at 37 C for two hr. The reaction mixture is further incubated for 20 min at 85 C, and probes are purified using two successive CHROMA SPIN+TE 30 columns (Clontech, Palo Alto Calif.). Purified probe is ethanol precipitated by diluting probe to 90 μl in DEPC-treated water, adding 2 μl 1 mg/ml glycogen, 60 μl 5 M sodium acetate, and 300 μl 100% ethanol. The probe is centrifuged for 20 min at 20,800× g, and the pellet is resuspended in 12 μl resuspension buffer, heated to 65C for five min, and mixed thoroughly. The probe is heated and mixed as before and then stored on ice. Probe is used in high density microarray-based hybridizations as described below.

Membrane-Based Hybridization

Membranes are pre-hybridized in hybridization solution containing 1% Sarkosyl and 1× high phosphate buffer (0.5 M NaCl, 0.1 M NaHPO₄, 5 mM EDTA, pH 7) at 55 C for two hr. The probe, diluted in 15 ml flesh hybridization solution, is then added to the membrane. The membrane is hybridized with the probe at 55 C for 16 hr. Following hybridization, the membrane is washed for 15 min at 25 C in 1 mM Tris (pH 8.0), 1% Sarkosyl, and four times for 15 min each at 25 C in 1 mM Tris (pH 8.0). To detect hybridization complexes, XOMAT-AR film (Eastman Kodak, Rochester N.Y.) is exposed to the membrane overnight at −70 C, developed, and examined visually.

Polymer Coated Slide-Based Hybridization

Probe is heated to 65 C for five min, centrifuged five min at 9400 rpm in a microfuge, and then 18 μl is aliquoted onto the microarray surface and covered with a coverslip. The micorarrays are transferred to a waterproof chamber having a cavity just slightly larger than a microscope slide. The chamber is kept at 100% humidity internally by the addition of 140 μl of 5×SSC in a corner of the chamber. The chamber containing the microarrays is incubated for about 6.5 hr at 60 C. The microarrays are washed for 10 min at 45 C in 1×SSC, 0.1% SDS, and three times for 10 min each at 45 C in 0.1×SSC, and dried.

Hybridization reactions are performed in absolute or differential hybridization formats. In the absolute hybridization format, probe from one sample is hybridized to the microarray elements, and signals are detected after hybridization complexes form. Signal strength correlates with probe mRNA levels in the sample. In the differential hybridization format, differential expression of a set of genes in two biological samples is analyzed Probes from the two samples are prepared and labeled with different labeling moieties. A mixture of the two labeled probes is hybridized to the microarray elements, and signals are examined under conditions in which the emissions from the two different labels are individually detectable. Elements on the microarray that are hybridized to equal numbers of probes derived from both biological samples give a distinct combined fluorescence (Shalon WO95/35505).

Hybridization complexes are detected with a microscope equipped with an Innova 70 mixed gas 10 W laser (Coherent, Santa Clara Calif.) capable of generating spectral lines at 488 nm for excitation of Cy3 and at 632 nm for excitation of Cy5. The excitation laser light is focused on the microarray using a 20× microscope objective (Nikon, Melville N.Y.). The slide containing the microarray is placed on a computer-controlled X-Y stage on the microscope and raster-scanned past the objective with a resolution of 20 micrometers. In the differential hybridization format, the two fluorophores are sequentially excited by the laser. Emitted light is split, based on wavelength, into two photomultiplier tube detectors (PMT R1477, Hamamatsu Photonics Systems, Bridgewater N.J.) corresponding to the two fluorophores. Filters positioned between the microarray and the photomultiplier tubes are used to separate the signals. The emission maxima of the fluorophores used are 565 nm for Cy3 and 650 nm for Cy5. The sensitivity of the scans is calibrated using the signal intensity generated by the yeast control mRNAs added to the probe mix. A specific location on the microarray contains a complementary DNA sequence, allowing the intensity of the signal at that location to be correlated with a weight ratio of hybridizing species of 1:100,000.

The output of the photomultiplier tube is digitized using a 12-bit RTI-835H analog-to-digital (A/D) conversion board (Analog Devices, Norwood Mass.) installed in an IBM-compatible PC computer. The digitized data are displayed as an image where the signal intensity is mapped using a linear 20-color transformation to a pseudocolor scale ranging from blue (low signal) to red (high signal). The data is also analyzed quantitatively. Where two different fluorophores are excited and measured simultaneously, the data are first corrected for optical crosstalk (due to overlapping emission spectra) between the fluorophores using the emission spectrum for each fluorophore. A grid is superimposed over the fluorescence signal image such that the signal from each spot is centered in each element of the grid. The fluorescence signal within each element is then integrated to obtain a numerical value corresponding to the average intensity of the signal.

1. A combination comprising primer sets selected from three biomarkers of insulin resistance (BIRs), wherein the primers are SEQ ID NOs:1-6.

2. A method for screening a subject for insulin resistance comprising:
a) obtaining a sample from the subject,
b) extracting mRNA from the sample,
c) performing real time quantitative PCR (RT-qPCR) on the sample using the combination of claim 1 to quantitate gene expression, and
d) comparing the amount of gene expression in the sample to the amount of gene expression in a sample from at least one normal subject, wherein differential expression between the samples indicates insulin resistance.
In one aspect of the invention, the samples are blood samples.

3. The method of claim 2 wherein the differential expression is at least a two-fold decrease in expression of at least one BIR.

4. The method of claim 2 wherein the expression in a sample from at least one normal age and gender matched subject further comprises a standard reference value for insulin sensitivity.

5. A method for monitoring IR status in an IR subject comprising:
a) obtaining a sample from the subject,
b) extracting mRNA from the sample,
c) performing real time quantitative PCR on the sample using the combination of claim 1 to assess gene expression, and
d) comparing gene expression in the sample to gene expression in a previous sample from the subject, wherein differential expression determines a change in IR status.

6. The method of claim 5 wherein a change in IR status indicates increased glucose tolerance or insulin sensitivity.

7. The method of claim 5 wherein a change in IR status indicates progression of IL

8. The method of claim 7 wherein a change in IR status is prognostic for the onset of IR-related conditions and non-insulin dependent diabetes mellitus.

9. The method of claim 5 wherein a change in IR status also indicates successful lifestyle intervention.

10. The method of claim 5 wherein a change in IR status following drug therapy indicates successful therapeutic intervention.

11. The method of claim 8 wherein IR-related conditions are selected from metabolic syndrome, obesity, atherosclerosis, peripheral vascular disease, hypertension, myocardial infarction and stroke.

12. A method for assessing therapeutic efficacy of a drug used to treat insulin resistance in a subject comprising:
a) administering a drug to the subject, and
b) comparing the amount of expression in a first sample taken from the subject prior to administration of the drug to the amount of expression in a second sample taken from the subject following administration of the drug, wherein a significant difference in expression indicates therapeutic efficacy.
